(19) United States
(12) Patent Application Publication
(10) Pub. No.: US 2013/0060271 A1
(43) Pub. Date: Mar. 7, 2013

(54) MULTI-CONFIGURABLE CORNEAL SURFACE MARKER

(71) Applicant: Murad A. Sunalp, Visalia, CA (US)

(72) Inventor: Murad A. Sunalp, Visalia, CA (US)

(21) Appl. No.: 13/225,308

(22) Filed: Sep. 2, 2011

(51) Int. Cl. A61F 9/007

(52) U.S. Cl. 606/166

(57) ABSTRACT

A corneal marking device for placing markings on the cornea of a patient. The device features a mounting member which is adapted for a hand gripping on one end and has a corneal marking component at the other. The marking component has a centrally located hub having an axial passage and has a plurality of marking fins radially extending from said hub. The leading edge of the fins of said marking component are adapted for placement of ink thereon which is communicated to the eye on contact. A light source communicating from the hub masks the approach of the device to the patient's eye diminishing patient flinching and eye movement. A two colored communication of the light source to the eye provides the user a visual reference to maintain the device in alignment with the patient's eye until contact.

This application claims priority to U.S. Provisional Application Serial Number 61/37996 filed on September 2, 2010 and is included herein in its entirety by reference. The invention herein disclosed relates generally to medical devices and tools adapted for employment with eye surgery and treatment. More particularly it relates to an instrument for medical professionals to employ for placing radial, horizontal, and vertical reference meridians upon the corneal surface to mark and to evaluate corneal topography.

### BACKGROUND OF THE INVENTION

1. Field of the Invention

2. Prior Art

Surgical treatment for many eye diseases and imperfections has evolved modernly into many different types of procedures. In carrying out a majority of such procedures it is extremely important that the surgeon be able to evaluate the corneal topography. Further, it is equally important that the eye of the patent be marked accurately to insure the most favorable outcome.

However, most marking components in the prior art fail to take into consideration, the angst of the patient from the marking device approaching their eyeball and the need to consistently align the axis of the radial marking device, with the center axis of the eye running through the center of the pupil. Further, most fail to take into consideration that a prone patient's eyes are in a different position than the same patient standing vertical and the need to make sure the radial lines are properly aligned with the eye axis is thus even more important.

Depending on the particular eye disease and the conditions of the patient's eye being treated, these markings, if accurately placed, provide the surgeon a map for determining and marking the appropriate arrangement of incisions. Small inaccuracies in planning can, and usually do lead to poor surgical outcomes. This is especially true in aligning the radial lines properly about the same axis as that of the eyeball being marked.

Consequently it is imperative that the surgeon have the appropriate tools to ascertain corneal topography as well as to mark the cornea accurately. Such accuracy should be easily repeatable and achieved while the user and/or surgeon is watching the marking fins approaching the surface of the patient's cornea to more accurately place them thereon. Such a device should allow the surgeon to align the axis of the eye through the pupil, consistently with the axis of the marking device around which the radial lines extend, without the need to constantly watch a level or other alignment device.

Further, it is equally imperative that the tools employed are easy to use and ideally easy to adapt to multiple different procedures, and patient positions, such that the surgeon does not require multiple tools for the same marking and measuring session. Finally, such a device should have components configured to help alleviate patient angst, and the possibility of eye movement when seeing the approaching marking fins, by masking the approach.

### SUMMARY OF THE INVENTION

The disclosed device and method for corneal marking provides a means to impart radial markings to the corneal surface of a patient from which the surgeon may evaluate a corneal topography for a current, and/or subsequent surgical procedures to the eye so marked. Further, it provides markings enabling a surgeon to reposition an eye flap, in its original registered position in front of the pupil, should the corneal hinge become detached or should the hinge not be cut and the flap become totally detached.

The device features one or a plurality of readily oriented finned markers having curved fin members extending away from the axis running though a central mounting hub. Each of the plurality of finned markers may have differently oriented fins having a curved marking surface, similar to the curve of an eyeball frontal surface, to thereby impart markings upon the corneal surface.

Each of the finned markers are engageable to the distal end of a mounting member adapted for hand holding by the surgeon. The mounting member is preferably straight in most cases, however in some cases, having it curved, or flexibly positionable may also be desired.

The markers may be provided in a kit with the plurality of finned markers having differently spaced and shaped radially extending fins, extending from and around a center axis of the mounting hub. As noted, the mounting member may be rigid in some cases and in other, a flexible mounting member.

If curved, the mounting member may be fixed in a curve or may be flexible to a curve. Formed in a straight alignment, the mounting member is adapted to engage with a light source which projects through the hub of the marker. The light so transmitted is preferably passed through an operatively positioned bubble level en route to the exit at the hub of the marker at the distal end of the mounting member where the finned marker is engaged. If the mounting member is formed in a curve, the light so transmitted will travel through a fiber optic cable positioned within the axial conduit of the mounting member and exit in the same fashion through the hub of the finned marker.

The mounting member is engaged at a proximal end as noted, to a hand grip or handle, which preferably has a light source thereon powered preferably by a power source in or engaged with the handle. The light source communicates light as noted to the distal end of the mounting member and through the hub of the marker or through a fiberoptic cable communicating through the axial passage of the mounting member to the distal end.

The light so transmitted is emitted from the distal end of the mounting member through the hub of the radially finned marker mounted thereon. The emitted light may be employed for a number or tasks such as a fixation light for centering the mounted radial fin on the determined patient's visual axis, or a corneal light for centering the fin marker on the axis extending from the pupil. Since the light is emitted from the center of the fin marker, it is easily employed for this purpose.

Projecting the light from the distal end through the hub has a secondary purpose which is most important. Patients are especially nervous to touching of their eye, even when it has been numbed. Consequently, they tend to flinch and move the eye at the sight of most approaching markers. The device herein, by emitting a bright light stream from the hub of the maker, blinds the patient to the sight of the approaching marker, and alleviates the potential of the patient flinching or moving their eye and causing problems with the marks achieved on the cornea.

The projected light is especially well adapted to provide the user with a visual means to determine that the axis of the radial finned marker, is substantially aligned with the visual axis of the patient extending through the pupil. A means to align the two axis is provided by projecting the light from the source, through the bubble level, to render it into a targeting means for the user which may be watched all the way to contact by the fins with the cornea.

The light from the source is projected through the bubble level, and if the level is perfectly level to earth, the light from the source travels both through the centrally located bubble, and the colorized fluid surrounding the centrally located bubble. Since the bubble is air and a lack of the fluid, it has no color. Light passing through the bubble is thus white, and on either side of the bubble, will be projected as the color of the fluid, which in this case is yellow for easy viewing on when communicated to the corneal surface.

In use, the surgeon or user need not look at the bubble to ascertain the bubble is level while approaching the patient's eye. Instead, by watching and ascertaining the continuous projection of a white light portion surrounded by colorized light, on the patient's eye, the surgeon is provided a means to visually ascertain alignment of the axis of the marking device, with the visual axis extending perpendicular to the center of the pupil of the patient's eye. This is because light projected through the level, will only allow the white light to travel through the mounting member or the fiber optic cable and to project on the cornea, if the level is perfectly level. On either side of the white light area, will be projected colorized areas. If the axis of the center of the hub of the marker, is not substantially parallel to a level surface, for a standing or sitting patient, the white light will not project on the pupil. If the axis of the hub of the marker is not perpendicular to true level for a prone patient, again, only the colorized light is projected upon the cornea of the patient. By watching the projected light and maintaining the white area of light centered on the pupil during approach of the marker, the user can continually see proper alignment with the visual axis of the eye running through the center of the pupil. As such, they need not watch any levels or markers during the process and can concentrate on centering the hub on the pupil to achieve the desired markings from the fins of the marker employed for marking the cornea with applied ink adapted to the task.

The size of hub and the radially extending fins can be provided in a standard size, for example one with a 12.5 mm diameter and 2.5 mm central zone for the hub, or it may be provided in a configuration adapted to a specific task which might require a non-standard configuration. Using the finned marker, orientation marks may be imparted to the cornea of the patient for procedures such as an LRI, a Toric IOL, a Toric ICL, a Lasik flap, for photograph of the cornea, for documentation for corneal lesions such as an ulcer and its location or size. The fin marker can also be employed for determination of pupil size mesopic/dilated, or capsulorhexis size for multifocal IOL.

The device is also configured for engagement of a secondary permanent or removable bubble lever component and can also be configured with attachment points for other components.

It is another object of this invention to provide a corneal marking device adapted for use for multiple procedures and employing easily engaged components which may be replaced depending upon the need. It is a further object to provide such a device, which projects a light stream to allow continuous alignment of the finned marker with the eye while concurrently obscuring the patient's view of the approaching marker to alleviate flinching and eye movement.

These together with other objects and advantages which become subsequently apparent reside in the details of the supplemental corneal marking device and system herein as more fully hereinafter described and claimed, reference being had to the accompanying drawings forming a part thereof, wherein like numerals refer to like parts throughout.

### BRIEF DESCRIPTION OF DRAWING FIGURE

FIG.1 depicts a perspective view of the radially configured finned marker engaged to the distal end of a flexible or permanently curved mounting member engaged to the handle.

FIG. 2 shows another view of the device of FIG. 1 but having a straight mounting member.

FIG. 3 shows an end view of the radially configured fin marker which is well adapted for determining pupil size, or capsulorhexis size for multifocal IOL.

FIG. 4 depicts a view of a typical finned marker such as in FIG. 3, in a perspective view.

FIG. 5 shows the fin marker of FIG. 4 from a side sectional view showing the central hub and center axis thereof.

FIG. 6 shows the light source traveling through the operatively positioned bubble level and exiting in colorized and white light streams.

FIG. 7 is another view of a light source of the device, traversing through a bubble level, and exiting in colorized and white light streams.

FIG. 8 is illustrative of the two different colorized light streams striking the patient's eyeball, only when the axis of the radially finned marker is aligned with the axis of the eyeball.

FIGS. 9 and 9a shows a round bubble level which may also be employed in place of the tubular bubble level of FIG. 7.

FIG. 10 shows the eyeball prior to the approach of the finned marker with the light streams exiting the hub.

FIG. 11 shows a mis-aligned hub where the light streams are only in one color of light.

FIG. 12 shows the eyeball of the patient with the finned marker approaching in proper alignment with a first colorized stream centered on the pupil and a secondary colorized light stream surrounding the first.

FIG. 13 depicts the markings from the curved blades of one favored mode of the finned marker having a fin with a secondary curve around the axis of the hub.

FIG. 14 depicts the marking from FIG. 13 on the eyeball.

FIG. 15 shows the marking of FIG. 14 on the cornea and cut flap in the cornea and showing the marking from the secondary curved fin providing means to register the flap to its original position should the hinge break or fail to be formed.

### DETAILED DESCRIPTION OF THE PREFERRED EMBODIMENTS OF THE INVENTION

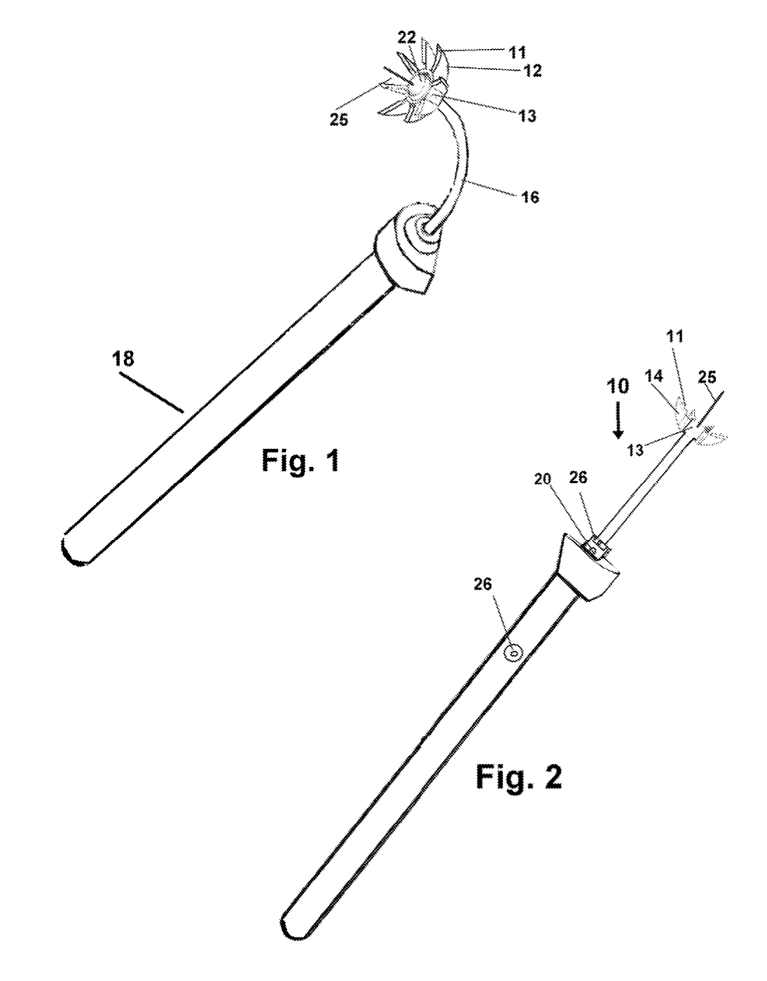

Referring now to the FIGS. 1-15, the radial eye marker device 10 which may be employed for corneal marking using ink or marking liquid placed on the curved frontal surfaces 11 of the curved mounting members 12 forming the radially deployed curved marking fins 14 or by making indents in the cornea therewith or both.

The device 10 may employ one or may have a kit featuring a plurality of marking components 13 having marking fins 14 formed by curved mounting members 12 radially deployed around a central hub 22 and having a curve to the frontal surfaces mirroring the curve of the corneal surface of an eyeball and in a manner adapted to the purpose intended. All the marking fins 14 are radially extending from the hub 22 which is engaged to the distal end of the curved or a flexible mounting member 16. The mounting member 16 may be fixed in a curve or may be flexible to a curve.

The mounting member 16 is engaged at a proximal end to a handle 18 with a communicating light source 20 therein powered preferably by a battery in or engaged with the handle 18. The handle mounted light source 20 communicates the light source 20 directly through an axial passage of the handle 18 or through a fiberoptic cable (not shown) communicating through the axial passage of the curved mounting member 16 to the distal end. The light from the light source 20 is thereby emitted from the distal end of the mounting member 16 and then through a circular hub 22 running along the axial passage 23 of the marking component 13 around which from which the individual curved mounting members 12 extend radially.

As noted, the emitted light through the aperture 22 may be employed for a number or tasks such as a fixation light for centering the mounted marking fin 14 on the visual axis, or as a corneal light for centering the marking component 14 and aligning it with the axis of the eyeball running through the pupil 15. Since the light source 20 is emitted from the hub 22 of the marking component 14 it is easily employed for this purpose.

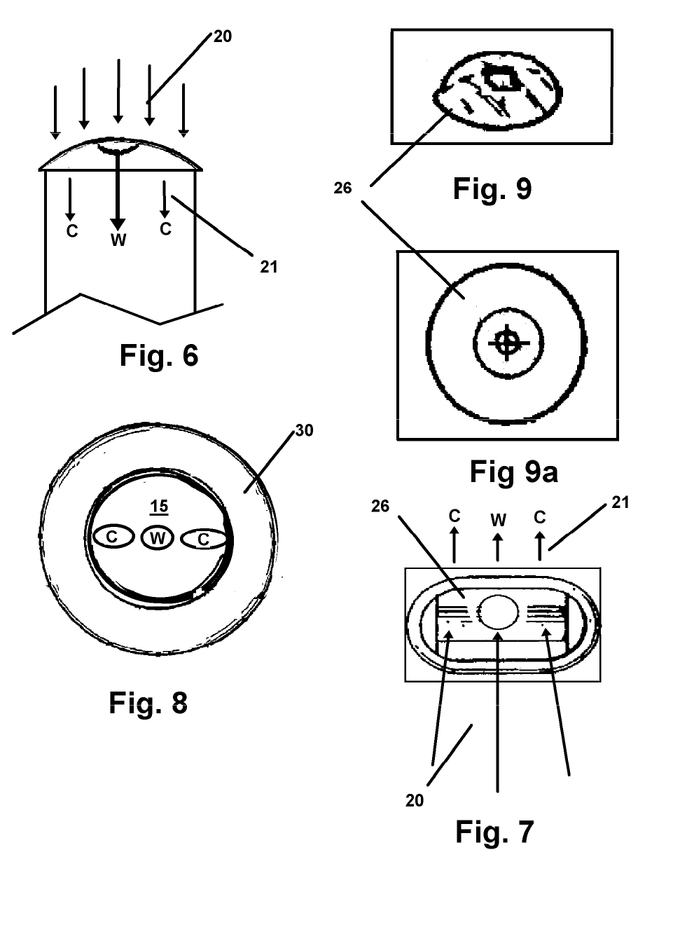

As shown in FIG. 6 the light source 20 is positioned so it travels through the operatively positioned bubble level 26 to form exiting light source 21 which is in a colorized stream of two colors of light, currently white light and a color of light of the fluid in the level 26. A tubular bubble level 26 may be employed as in FIG. 7 in a particularly favored mode of the device 10 as it allows for alignment of the axis 25 of the hub 22 with the axis extending from the pupil 15 whether the patient is sitting, standing, or laying on their back.

This means for axis alignment is shown in FIG. 8 which is illustrative of the two different colorized light streams C and W, striking the patient's eyeball. Both streams C and W only are visible when the axis 25 of the radially finned marker 14 is aligned with the axis 29 of the eyeball 30. FIGS. 9 and 9a shows a round bubble level 26 which may also be employed in place of the tubular bubble level 26 which is more preferred.

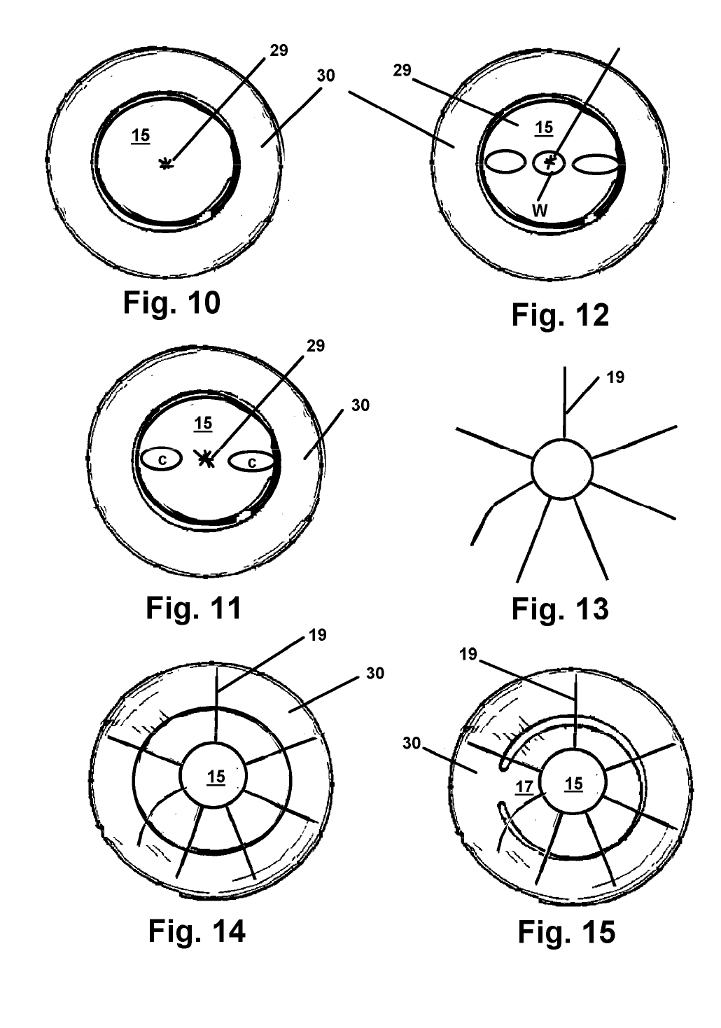

In FIG. 10 is seen the eyeball 30 prior to the approach of the finned marker 13 with the light streams 21 exiting the hub 22. If there is mis alignment of the axis 25 and axis 29, such can be seen in FIG. 11 which shows a mis-alignment where the light streams 21 are only in one color C, of light 21. However, if the axis are in alignment, FIG. 12 shows the eyeball 30 of the patient with the finned marker 13 approaching in proper alignment with a first colorized stream W, centered on the pupil 15 and a secondary colorized light stream C surrounding the first. This can be continuously viewed by the doctor until contact with the eyeball by the finned marker 13.

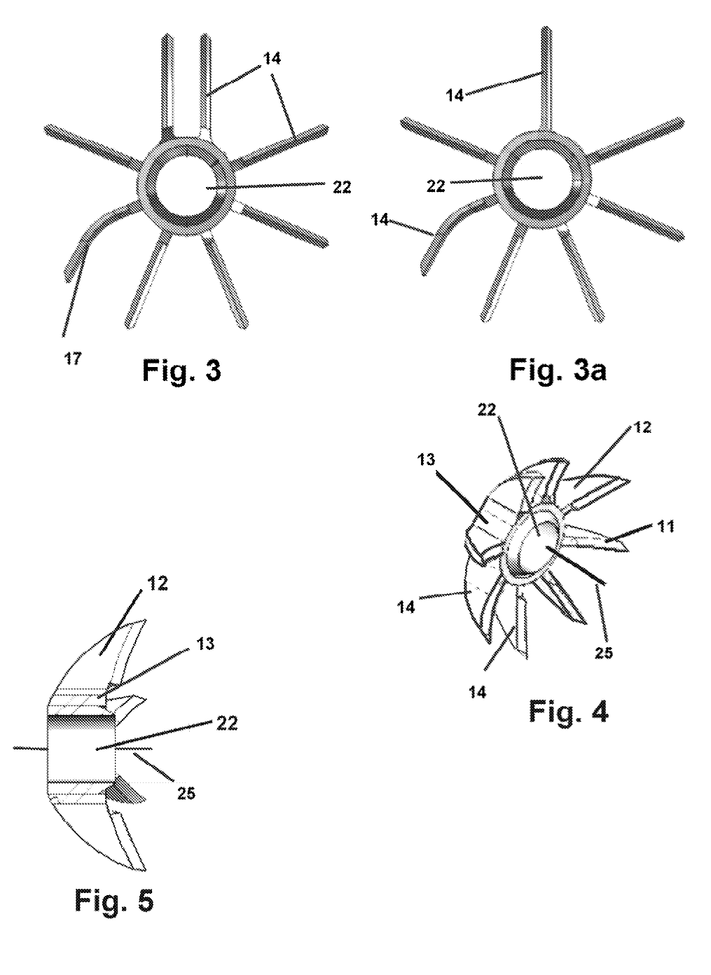

As shown in FIG. 13 the markings 19 from the ink or indents caused by the curved fins front surfaces 11, of one favored mode of the finned marker 13, have markings 19 from one fin 17 (FIGS. 3 and 3a), with a secondary curve around the axis 25 of the hub 22. When placed on the eyeball as in FIG. 13 and cut flap 19 in the cornea, the marking from the secondary curved fin 17 provide means to register the flap 17 to its original position both horizontally and vertically around the axis 29, should the hinge break or fail to be formed and the flap 17 be detached. Alignment of all of the lines is easier to discern with the curved marking shown in FIGS. 13 and 14.

Finally, the light 21 exiting the hub 22 is projected directly to the pupil 15 of the patient, and provides a means to mask the approach of the marker 13 until it contacts the eye. Thus the patient cannot see the approaching marker 13 and does not flinch or move the eye before a contact.

The bubble level 26 may be situated at the distal end of the mounting member 16 between the mounting member 16 and the hub 22 for best results, however it may be positioned in the light stream 20 anywhere that it will provide two different exiting light streams 21 with different colors which will only show on the eye if the bubble level 26 is level, and hence the axis of the marker 13 and eye align.

While all of the fundamental characteristics and features of the device herein, and employment thereof have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the disclosed system and apparatus as defined by the following claims.

What is claimed is:

1. A corneal marking apparatus comprising:
a mounting member adapted at a proximal end for a hand gripping and having a distal end;
a corneal marking component having a centrally located hub having an axial passage therethrough having a center axis, said hub having a plurality of marking fins radially extending from said hub;
a leading edge of said fins of said marking component adapted for placement of ink thereon;
means for engagement of a first side of said corneal marking component to said distal end of said mounting member;
a light source communicating through said hub from said first side and exiting said hub at a second end opposite said first side; and
said light source providing means to mask an approach of said corneal marking component to an eye of a patient through communication of said light source exiting said hub, with said patient's eye thereby impairing a visualization by said patient of said marking component until a contact with said eye is substantially achieved.

2. The corneal marking apparatus of claim 1 additionally comprising:
said leading edge having a curved edge, said curved edge being complimentary to a curved exterior surface of said eye.

3. The corneal marking apparatus of claim 1, additionally comprising:
at least one of said fins having a secondary curve in a direction around said axis of said hub.

4. The corneal marking apparatus of claim 2, additionally comprising:
at least one of said fins having a secondary curve in a direction around said axis of said hub.

5. The corneal marking apparatus of claim 1, additionally comprising:
a bubble level having fluid of fluid therein of a first color;
said bubble level having a substantially level position wherein a bubble in said fluid is within a central area of said fluid in said level;
said bubble having a secondary position wherein said bubble is outside said central area of said fluid;
said light source communicating through said bubble level prior to exiting said hub at a second end;
said communication of said light source exiting said hub, with said patient's eye being solely in said first color when said bubble is in said secondary position;
said communication of said light source exiting said hub with said patient's eye having second colorized area of a second color abutted by said first color on opposite sides of said second colorized area, when said bubble is in said level position; and
a manipulation of said apparatus to maintain said second colorized area in said communication with said patient's eye, until said contact with said eye, providing means to align said center axis with a visual axis of said patient, until said contact is achieved.

6. The corneal marking apparatus of claim 2, additionally comprising:
a bubble level having fluid of fluid therein of a first color;
said bubble level having a substantially level position wherein a bubble in said fluid is within a central area of said fluid in said level;
said bubble having a secondary position wherein said bubble is outside said central area of said fluid;
said light source communicating through said bubble level prior to exiting said hub at a second end;
said communication of said light source exiting said hub, with said patient's eye being solely in said first color when said bubble is in said secondary position;
said communication of said light source exiting said hub with said patient's eye having second colorized area of a second color abutted by said first color on opposite sides of said second colorized area, when said bubble is in said level position; and
a manipulation of said apparatus to maintain said second colorized area in said communication with said patient's eye, until said contact with said eye, providing means to align said center axis with a visual axis of said patient, until said contact is achieved.

7. The corneal marking apparatus of claim 3, additionally comprising:
a bubble level having fluid of fluid therein of a first color;
said bubble level having a substantially level position wherein a bubble in said fluid is within a central area of said fluid in said level;
said bubble having a secondary position wherein said bubble is outside said central area of said fluid;
said light source communicating through said bubble level prior to exiting said hub at a second end;
said communication of said light source exiting said hub, with said patient's eye being solely in said first color when said bubble is in said secondary position;
said communication of said light source exiting said hub with said patient's eye having second colorized area of a second color abutted by said first color on opposite sides of said second colorized area, when said bubble is in said level position; and
a manipulation of said apparatus to maintain said second colorized area in said communication with said patient's eye, until said contact with said eye, providing means to align said center axis with a visual axis of said patient, until said contact is achieved.

8. The corneal marking apparatus of claim 4, additionally comprising:
a bubble level having fluid of fluid therein of a first color;
said bubble level having a substantially level position wherein a bubble in said fluid is within a central area of said fluid in said level;
said bubble having a secondary position wherein said bubble is outside said central area of said fluid;
said light source communicating through said bubble level prior to exiting said hub at a second end;
said communication of said light source exiting said hub, with said patient's eye being solely in said first color when said bubble is in said secondary position;
said communication of said light source exiting said hub with said patient's eye having second colorized area of a second color abutted by said first color on opposite sides of said second colorized area, when said bubble is in said level position; and
a manipulation of said apparatus to maintain said second colorized area in said communication with said patient's eye, until said contact with said eye, providing means to align said center axis with a visual axis of said patient, until said contact is achieved.
